(19) United States
(12) Patent Application Publication
(10) Pub. No.: US 2022/0137069 A1
(43) Pub. Date: May 5, 2022

(54) S100A12 AS BLOOD BIOMARKER FOR THE NON-INVASIVE DIAGNOSIS OF ENDOMETRIOSIS

(71) Applicant: Roche Diagnostics Operations, Inc., Indianapolis, IN (US)

(72) Inventors: Aikaterini GEORGOPOULOU, Luzern (CH); Feliz GRUENEWALD, Penzberg (DE); Martin HUND, Horw (CH); Martin KLAMMER, Muenchen (DE); Heike WEGMEYER, München (DE)

(21) Appl. No.: 17/575,202

(22) Filed: Jan. 13, 2022

Related U.S. Application Data
(63) Continuation of application No. PCT/EP2020/070426, filed on Jul. 20, 2020.

(30) Foreign Application Priority Data
Jul. 22, 2019 (EP) 19187477.5

(51) Int. Cl. G01N 33/68

(52) U.S. Cl. CPC G01N 33/6893; G01N 2800/364

(57) ABSTRACT

The present invention relates to methods of assessing whether a patient has endometriosis or is at risk of developing endometriosis, to methods of selecting a patient for therapy, and method of monitoring a patient suffering from endometriosis or being treated for endometriosis by determining the amount or concentration of S100A12 in a sample of the patient, and comparing the determined amount or concentration to a reference.

### CROSS-REFERENCE TO RELATED APPLICATIONS

This application is a continuation of International Application No. PCT/EP2020/070426 filed Jul. 20, 2020, which claims priority to European Application 19187477.5 filed Jul. 22, 2019, the disclosures of which are hereby incorporated by reference in their entirety.

The present invention relates to methods of assessing whether a patient has endometriosis or is at risk of developing endometriosis, to methods of selecting a patient for therapy, and methods of monitoring a patient suffering from endometriosis or being treated for endometriosis, by determining the amount or concentration of S100A12 in a sample of the patient, and comparing the determined amount or concentration to a reference.

### BACKGROUND OF THE INVENTION

Endometriosis is defined as the presence of endometrial glands and stroma-like lesions outside of the uterus. The lesions can be peritoneal lesions, superficial implants or cysts on the ovary, or deep infiltrating disease. Endometriosis affects 5-8% of all women of reproductive age and 70% of women with chronic pelvic pain. The prevalence of endometriosis has been estimated at 176 million women worldwide (Adamson et al. J Endometr. 2010; 2: 3-6). For many of these women there is often a delay in diagnosis of endometriosis resulting in unnecessary suffering and reduced quality of life. In patients aged 18-45 years, there is a delay of 7-10 years. As most women with endometriosis report the onset of symptoms during adolescence, early referral, diagnosis, identification of disease and treatment may mitigate pain preventing disease progression. Barriers to early diagnosis include the high cost of diagnosis and treatment in adolescent patients and presentation of confounding symptoms such as cyclic and non-cyclic pain (Parasar et al. Curr Obstet Gynecol Rep. 2017; 6: 34-41).

Gold standard for the diagnosis of endometriosis is laparoscopic visualization and subsequent histological confirmation. Until now, there are no non-invasive methods for the diagnosis of endometriosis (Hsu et al. Clin Obstet Gynecol 2010: 53: 413-419). During a diagnostic laparoscopy, a gynaecologist with training and skills in laparoscopic surgery for endometriosis should perform a systematic inspection of the pelvis (NICE guideline NG73, 2017). Surgical visualization requires good expertise, training and skills for reliable diagnosis. The fact that laparoscopic surgery is needed for diagnosis, which is avoided by doctors as long as possible, leads to a 7-10 year delay in diagnosis. The lack of a non-invasive diagnostic test significantly contributes to the long delay between onset of the symptoms and definitive diagnosis of endometriosis (Signorile and Baldi. J Cell Physiol 2014; 229: 1731-1735). Thus there is an unmet medical need for a non-invasive test for the diagnosis of endometriosis, in particular for the diagnosis of early, minimal and mild endometriosis (revised American Society for Reproductive Medicine rASRM stages I-II).

Adenomyosis is defined as infiltration of benign endometrial glands and stroma into the myometrium, the outer muscle layer of the uterus. There are three distinct adenomyosis subtypes, depending on the morphology and location: internal adenomyosis, external adenomyosis and adenomyomas. Internal adenomyosis can be further classified into focal, diffuse and superficial adenomyosis (Bazot M et al. Fertil Steril. 2018; 109:389-397). Adenomyosis can also be classified based on Magnetic Resonance Imaging (MRI) findings, according to whether it is located in the outer or the inner uterine layer, into 4 subtypes I-IV. Common signs and symptoms of adenomyosis include heavy bleeding during menstruation (menorrhagia) and in-between menstrual periods (metrorrhagia), dysmenorrhea, chronic pelvic pain, dyspareunia and infertility (Chapron C et al. Hum Reprod Update. 2020; 26:392-411).

Transvaginal ultrasound (TVUS) and Magnetic Resonance Imaging (MRI) are currently used to diagnose adenomyosis, with an overall sensitivity/specificity of 83.8%, 63.9% and 77.3%, 89.8% respectively. Among the different adenomyosis types, diffuse adenomyosis is more difficult to detect by imaging and requires an experienced sonographer. Also, access to imaging equipment is limited, especially among primary care healthcare professionals, and requires trained staff and specialized resources. Therefore, a non-invasive blood-based test would allow medical assessment of adenomyosis without a need for imaging device, reduce the inter-operator variability and enable a more standardized diagnosis of the condition (Chapron C et al. Hum Reprod Update. 2020; 26:392-411).

Non-invasive diagnosis of endometriosis would allow earlier diagnosis and treatment, with the potential to improve quality of life and reduce the societal costs related to endometriosis, and has therefore been selected as a research priority by the World Endometriosis Society (WES) and the World Endometriosis Research Foundation (WERF) (Fassbender et al., Springer, Peripheral Blood Biomarkers for Endometriosis. 2017). Thus, a non-invasive tool to diagnose endometriosis could facilitate earlier diagnosis and intervention that could ultimately improve quality of life and preserve fertility (Parasar et al. Curr Obstet Gynecol Rep. 2017; 6: 34-41).

Blood biomarkers are essential to reduce the time delay of diagnosing endometriosis that require laparoscopy. CA-125 is one of the most commonly used blood biomarkers, however, it's diagnostic utility is limited to endometriosis rASRM stages III and IV (Nisenblat et al., Cochrane Database of Systematic Reviews. 2016; 5: CD012179).

In endometriosis, elevated S100A12 levels have been reported in endometrial stromal cells (ESCs) derived from ectopic endometrium (lesions) of endometriosis patients compared to ESCs from healthy subjects. Higher mRNA and protein levels of S100A12 (EN-RAGE) were detected in primary endometrial stromal cells (ESCs) isolated from ectopic endometrium (lesions) of patients with endometriosis (advanced disease) compared to ESCs isolated from endometrium of healthy women (p<0.001) (Sharma et al. Int J Gynaecol Obstet. 2010; 110: 199-202). Higher S100A12 levels were detected in the ESCs from endometriotic patients upon incubation with Lipopolysaccharides (LPS). Treatment of these cells with atorvastatin, a compound with anti-inflammatory effects, led to a reduction of LPS-mediated S100A12 expression, suggesting that this ligand plays a role in inflammatory responses within ESCs (Sharma et al. Fertil Steril. 2010; 94(5): 1639-1646).

### SUMMARY OF THE INVENTION

In a first aspect, the present invention relates to a method of assessing whether a patient has endometriosis or is at risk of developing endometriosis, comprising determining the amount or concentration of S100A12 in a sample of the patient, and comparing the determined amount or concentration to a reference.

In a second aspect, the present invention relates to a method of selecting a patient for therapy (in particular drug-based therapy or surgical therapy (laparoscopy) of endometriosis, comprising determining the amount or concentration of S100A12 in a sample of the patient, and comparing the determined amount or concentration to a reference.

In a third aspect, the present invention relates to a method of monitoring a patient suffering from endometriosis or being treated for endometriosis, comprising determining the amount or concentration of S100A12 in a sample of the patient, and comparing the determined amount or concentration to a reference.

### LIST OF FIGURES

FIGS. 1A-1C: Receiver Operator Curve (ROC) analyses for single biomarkers (FIG. 1A) S100A12, (FIG. 1B) CA-125 and (FIG. 1C) clinical symptom dysmenorrhea.

x-axis=specificity, y-axis=sensitivity

FIGS. 2A-2C: Receiver Operator Curve (ROC) analyses for combination of biomarkers and combination of biomarkers and clinical symptoms.

x-axis=specificity, y-axis=sensitivity

FIGS. 3A & 3B: Box plots of S100A12 in endometriosis cases (Caes) controls (FIG. 3A) and S100A12 in cases rASRM stage I-II (G1/2), rASRM stages III-IV (G3/4) and Controls (FIG. 3B).

FIGS. 4A-4C: Box plots and ROC curves of serum levels of S100A12 in adenomyosis cases (rASRM stages 1-IV) and controls.

FIGS. 5A-5C: Box plots and ROC curves of serum levels of CA-125 in adenomyosis cases (rASRM stages I-IV) and controls.

FIGS. 6A-6C: Box plots and ROC curves of combination of serum levels of S100A12 and CA-125 in endometriosis cases (rASRM stages I-IV) and controls.

FIGS. 7A-7C: Box plots and ROC curves of serum levels of S100A12 in endometriosis cases (rASRM stages I-II) and controls.

FIGS. 8A-8C: Box plots and ROC curves of serum levels of CA-125 in endometriosis cases (rASRM stages I-II) and controls.

FIGS. 9A & 9B: Box plots and ROC curves of serum levels of S100A12 in adenomyosis cases and controls.

FIGS. 10A & 10B: Box plots and ROC curves of serum levels of CA-125 in adenomyosis cases and controls.

### LIST OF TABLES

Table 1: Diagnostic performance of biomarker S100A12 and biomarker combinations in women with endometriosis and controls.

Table 2: Cut-off for combination of various biomarkers to predict endometriosis based on multivariable logistic regression analysis and Youden's index.

Table 3: Diagnostic performance of single biomarkers S100A12 and CA-125 and biomarker combination in endometriosis cases (rASRM stages I-IV) and controls.

Table 4: Diagnostic performance of single biomarkers S100A12 and CA-125 in endometriosis cases (rASRM stages I-II) and controls.

Table 5: Diagnostic performance of single biomarkers S100A12 and CA-125 in adenomyosis cases and controls.

### DETAILED DESCRIPTION OF THE INVENTION

We show for the first time that S100A12 measured in blood is increased in women with endometriosis compared to controls. The blood levels of S100A12 are already increased in early endometriosis rASRM stages I and II (revised American Society for Reproductive Medicine) reflecting minimal and mild endometriosis, respectively. We also demonstrate that combining S100A12 with dysmenorrhea (menstrual cycle-dependent pain) and/or the biomarker CA-125 increased the diagnostic performance for distinguishing women with endometriosis from control women without endometriosis. S100A12 alone or in combination with dysmenorrhea/lower abdominal pain and/or CA-125 has the advantage of a non-invasive blood based test that identifies women with early stages of endometriosis (rASRM stages I and II) that is currently not possible with a non-invasive test.

### Definitions

The word “comprise”, and variations such as “comprises” and “comprising”, will be understood to imply the inclusion of a stated integer or step or group of integers or steps but not the exclusion of any other integer or step or group of integers or steps.

As used in this specification and the appended claims, the singular forms “a”, “an”, and “the” include plural referents, unless the content clearly dictates otherwise.

Concentrations, amounts, and other numerical data may be expressed or presented herein in a “range” format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “150 mg to 600 mg” should be interpreted to include not only the explicitly recited values of 150 mg to 600 mg, but to also include individual values and sub-ranges within the indicated range. Thus, included in this numerical range are individual values such as 150, 160, 170, 180, 190, . . . 580, 590, 600 mg and sub-ranges such as from 150 to 200, 150 to 250, 250 to 300, 350 to 600, etc. This same principle applies to ranges reciting only one numerical value. Furthermore, such an interpretation should apply regardless of the breadth of the range or the characteristics being described.

The term “about” when used in connection with a numerical value is meant to encompass numerical values within a range having a lower limit that is 5% smaller than the indicated numerical value and having an upper limit that is 5% larger than the indicated numerical value.

The term “indicator” as used herein, refers to a sign or signal for a condition or is used to monitor a condition. Such a “condition” refers to the biological status of a cell, tissue or organ or to the health and/or disease status of an individual. An indicator may be the presence or absence of a molecule, including but not limited to peptide, protein, and nucleic acid, or may be a change in the expression level or pattern of such molecule in a cell, or tissue, organ or individual. An indicator may be a sign for the onset, development or presence of a disease in an individual or for the further progression of such disease. An indicator may also be a sign for the risk of developing a disease in an individual.

In the context of present invention, the term “biomarker” refers to a substance within a biological system that is used as an indicator of a biological state of said system. In the art, the term “biomarker” is sometimes also applied to means for the detection of said endogenous substances (e.g. antibodies, nucleic acid probes etc, imaging systems). In the context of present invention, the term “biomarker” shall be only applied for the substance, not for the detection means. Thus, biomarkers can be any kind of molecule present in a living organism, such as a nucleic acid (DNA, mRNA, miRNA, rRNA etc.), a protein (cell surface receptor, cytosolic protein etc.), a metabolite or hormone (blood sugar, insulin, estrogen, etc.), a molecule characteristic of a certain modification of another molecule (e.g. sugar moieties or phosphoryl residues on proteins, methyl-residues on genomic DNA) or a substance that has been internalized by the organism or a metabolite of such a substance.

“S100A12”, also known as Calgranulin C, MRP6 or EN-RAGE (Extracellular Newly identified Receptor for Advanced Glycation Endproducts binding protein) is a 12 kilodalton calcium-binding protein that belongs to the S100 protein family. S100 proteins are exclusively expressed in vertebrates, display cell-specific distribution and regulate a large variety of intracellular and extracellular activities. The name S100 is derived from the fact that these proteins are 100% soluble in ammonium sulfate at physiological pH. In healthy individuals, expression of S100A12 has been reported in tissues and organs where resident neutrophils and monocytes are present, such as the spleen and lung. S100A12 is predominantly found in the cell cytosol but translocation to the membrane and the cytoskeletal components is observed upon elevated intracellular calcium levels. Intracellular S100A12 actionsinclude cytokine production and induction of oxidative stress. When secreted extracellularly, S100A12 contributes to the activation of innate immune response, acting as an alarmin or Danger-Activated Molecular Pattern (DAMP) (Donato Int J Biochem Cell Biol 2001; 33:637-668). S100A12 is a ligand of RAGE (receptor for advanced glycation endproducts). Binding of S100A12 to RAGE leads to activation of the MAPK/NF-kB pathway and the production of inflammatory cytokines such as tumor necrosis factor (TNF)-α and Interleukin (IL)-1β within immune cells (i.e. monocytes/macrophages/microglia, neutrophils and lymphocytes) (Yang et al. J Allergy Clin Immunol. 2007; 119 (1): 106-114). Increased levels of serum S100A12 were reported in inflammatory bowel disease, rheumatoid/psoriatic arthritis and cystic fibrosis where chronic inflammation plays a key role (Meijer et al. Int J Inflam. 2012:907078 doi:10.1155/2012/907078). “CA-125”, the Carbohydrate antigen 125, sometimes named as Cancer Antigen 125 or Tumor Antigen 125, is a mucin-type glycoprotein, produced by the MUC16 gene, and associated with the cellular membrane. CA-125 is a biomarker for epithelial cell ovarian cancer being derived from coelomic epithelia including the endometrium, fallopian tube, ovary, and peritoneum. Diagnostic use of CA-125 is limited to endometriosis stages III and IV (moderate and severe endometriosis) with moderate sensitivity.

“Symptoms” of a disease are implication of the disease noticeable by the tissue, organ or organism having such disease and include but are not limited to pain, weakness, tenderness, strain, stiffness, and spasm of the tissue, an organ or an individual. “Signs” or “signals” of a disease include but are not limited to the change or alteration such as the presence, absence, increase or elevation, decrease or decline, of specific indicators such as biomarkers or molecular markers, or the development, presence, or worsening of symptoms. Symptoms of pain include, but are not limited to an unpleasant sensation that may be felt as a persistent or varying burning, throbbing, itching or stinging ache.

The term “disease” and “disorder” are used interchangeably herein, referring to an abnormal condition, especially an abnormal medical condition such as an illness or injury, wherein a tissue, an organ or an individual is not able to efficiently fulfil its function anymore. Typically, but not necessarily, a disease is associated with specific symptoms or signs indicating the presence of such disease. The presence of such symptoms or signs may thus, be indicative for a tissue, an organ or an individual suffering from a disease. An alteration of these symptoms or signs may be indicative for the progression of such a disease. A progression of a disease is typically characterised by an increase or decrease of such symptoms or signs which may indicate a “worsening” or “bettering” of the disease. The “worsening” of a disease is characterised by a decreasing ability of a tissue, organ or organism to fulfil its function efficiently, whereas the “bettering” of a disease is typically characterised by an increase in the ability of a tissue, an organ or an individual to fulfil its function efficiently. A tissue, an organ or an individual being at “risk of developing” a disease is in a healthy state but shows potential of a disease emerging. Typically, the risk of developing a disease is associated with early or weak signs or symptoms of such disease. In such case, the onset of the disease may still be prevented by treatment. Examples of a disease include but are not limited to inflammatory diseases, infectious diseases, cutaneous conditions, endocrine diseases, intestinal diseases, neurological disorders, joint diseases, genetic disorders, autoimmune diseases, traumatic diseases, and various types of cancer.

“Endometriosis” is a chronic, hormone-dependent, inflammatory disease that is characterized by lesions of endometrial-like tissue outside of the uterus. Clinical presentation of endometriosis varies significantly from patient to patient. Endometriosis patients often present with symptoms such as intermenstrual bleeding, painful periods (dysmenorrhea), painful intercourse (dyspareunia), painful defecation (dyschezia) and painful urination (dysuria). Pelvic pain due to endometriosis is usually chronic (lasting ≥6 months) and is associated with dysmenorrhea (in 50 to 90% of cases), dyspareunia, deep pelvic pain, and lower abdominal pain with or without back and loin pain. The pain can occur unpredictably and intermittently throughout the menstrual cycle or it can be continuous, and it can be dull, throbbing, or sharp, and exacerbated by physical activity. Bladder- and bowel-associated symptoms (nausea, distention, and early satiety) are typically cyclic. Pain often worsens over time and may change in character; infrequently, women report burning or hypersensitivity, symptoms that are suggestive of a neuropathic component. Often, endometriosis can be asymptomatic, only coming to a clinician's attention during evaluation for infertility (Sinaii et al. Fertil Steril. 2008; 89(3): 538-545). In women with endometriosis, there is a reduced monthly fecundity rate (2-10%) compared with fertile couples (15-20%). Although endometriosis impairs fertility, it does not usually completely prevent conception (Fadhlaoui et al. Front Surg. 2014; 1: 24).

The most commonly affected sites of endometriosis are the pelvic organs and peritoneum, although other parts of the body such as the lungs are occasionally affected. The extent of the disease varies from a few, small lesions on otherwise normal pelvic organs to large, ovarian endometriotic cysts (endometriomas) and/or extensive fibrosis and adhesion formation causing marked distortion of pelvic anatomy. Based on the location, endometriotic lesions can be classified into peritoneal endometriosis, ovarian endometriotic cysts (endometrioma), deep nodules (deep infiltrating endometriosis), and adenomyosis (Kennedy et al. Hum Reprod. 2005; 20(10): 2698-2704).

The term “rASRM stage” or “rASRM staging” refers to the revised classification system established by the American Society for Reproductive Medicine (ASRM) describing the severity of endometriosis based on the findings at surgery (laparoscopy). The classification is based on the morphology of peritoneal and pelvic implants such as red, white and black lesions, percentage of involvement of each lesion should be included. Number, size, and location endometrial implants, plaques, endometriomas and adhesions should be noted. Endometriosis in bowel, urinary tract, fallopian tube, vagina, cervix, skin, or other locations should be documented per ASRM guidelines. Stages of endometriosis according to ASRM guidelines are stage I, II, III, and IV determined based on the point scores and correspond to minimal, mild, moderate and severe endometriosis. The rASRM stages I & II endometriosis (minimal to mild endometriosis) are defined by superficial peritoneal endometriosis, possible presence of small deep lesions, absence of endometrioma and/or mild filmy adhesion. The rASRM stages III and IV endometriosis (moderate to severe endometriosis) are defined by the presence of superficial peritoneal endometriosis, deep infiltrating endometriosis with moderate to extensive adhesions between the uterus and bowels and/or endometrioma cysts with moderate to extensive adhesions involving the ovaries and tubes.

The term “VAS”, the Visual Analog Scale, is an instruments to assess the intensity of pain. The VAS consists of a 10-cm long horizontal line with its extremes marked as ‘no pain’ and ‘worst pain imaginable’. Each patient ticks her pain level on the line and the distance from ‘no pain’ on the extreme left to the tick mark is measured in centimeters, yielding a pain score from 0 to 10. ‘No pain’ corresponds to a pain score of 0, ‘worst pain imaginable’ corresponds to a pain score of 10. In women with endometriosis dysmenorrhea is associated with the highest perception of pain with a mean VAS score of about 6 (Cozzolino et al. Rev Bras Ginecol Obstet 2019; 41(3): 170-175).

As used herein, a “patient” means any mammal, fish, reptile or bird that may benefit from the diagnosis, prognosis or treatment described herein. In particular, a “patient” is selected from the group consisting of laboratory animals (e.g. mouse, rat, rabbit, or zebrafish), domestic animals (including e.g. guinea pig, rabbit, horse, donkey, cow, sheep, goat, pig, chicken, camel, cat, dog, turtle, tortoise, snake, lizard or goldfish), or primates including chimpanzees, bonobos, gorillas and human beings. It is particularly preferred that the “patient” is a human being.

The term “sample” or “sample of interest” are used interchangeably herein, referring to a part or piece of a tissue, organ or individual, typically being smaller than such tissue, organ or individual, intended to represent the whole of the tissue, organ or individual. Upon analysis a sample provides information about the tissue status or the health or diseased status of an organ or individual. Examples of samples include but are not limited to fluid samples such as blood, serum, plasma, synovial fluid, urine, saliva, and lymphatic fluid, or solid samples such as tissue extracts, cartilage, bone, synovium, and connective tissue. Analysis of a sample may be accomplished on a visual or chemical basis. Visual analysis includes but is not limited to microscopic imaging or radiographic scanning of a tissue, organ or individual allowing for morphological evaluation of a sample. Chemical analysis includes but is not limited to the detection of the presence or absence of specific indicators or alterations in their amount, concentration or level. The sample is an in vitro sample, it will be analyzed in vitro and not transferred back into the body.

The term “amount” as used herein encompasses the absolute amount of a biomarker as referred to herein, the relative amount or concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts measured from biological read out systems in response to the peptides or intensity signals obtained from specifically bound ligands. It is to be understood that values correlating to the aforementioned amounts or parameters can also be obtained by all standard mathematical operations.

The term “comparing” as used herein refers to comparing the amount of the biomarker in the sample from the subject with the reference amount of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer-assisted. Thus, the comparison may be carried out by a computing device. The value of the measured or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the measured amount may be compared to values corresponding to suitable references which are stored in a database by a computer program. The computer program may further evaluate the result of the comparison, i.e. automatically provide the desired assessment in a suitable output format. For a computer-assisted comparison, the value of the measured amount may be compared to values corresponding to suitable references which are stored in a database by a computer program. The computer program may further evaluate the result of the comparison, i.e. automatically provides the desired assessment in a suitable output format.

The expression “comparing the amount or concentration determined to a reference” is merely used to further illustrate what is obvious to the skilled artisan anyway. A reference concentration is established in a control sample

The term “reference sample” or “control sample” as used herein, refers to a sample which is analysed in a substantially identical manner as the sample of interest and whose information is compared to that of the sample of interest. A reference sample thereby provides a standard allowing for the evaluation of the information obtained from the sample of interest. A control sample may be derived from a healthy or normal tissue, organ or individual, thereby providing a standard of a healthy status of a tissue, organ or individual. Differences between the status of the normal reference sample and the status of the sample of interest may be indicative of the risk of disease development or the presence or further progression of such disease or disorder. A control sample may be derived from an abnormal or diseased tissue, organ or individual thereby providing a standard of a diseased status of a tissue, organ or individual. Differences between the status of the abnormal reference sample and the status of the sample of interest may be indicative of a lowered risk of disease development or the absence or bettering of such disease or disorder. A reference sample may also be derived from the same tissue, organ, or individual as the sample of interest but has been taken at an earlier time point. Differences between the status of the earlier taken reference sample and the status of the sample of interest may be indicative of the progression of the disease, i.e. a bettering or worsening of the disease over time.

The control sample may be an internal or an external control sample. An internal control sample is used, i.e. the marker level(s) is(are) assessed in the test sample as well as in one or more other sample(s) taken from the same subject to determine if there are any changes in the level(s) of said marker(s). For an external control sample the presence or amount of a marker in a sample derived from the individual is compared to its presence or amount in an individual known to suffer from, or known to be at risk of, a given condition; or an individual known to be free of a given condition, i.e., “normal individual”.

It will be appreciated by the skilled artisan that such external control sample may be obtained from a single individual or may be obtained from a reference population that is age-matched and free of confounding diseases. Typically, samples from 100 well-characterized individuals from the appropriate reference population are used to establish a “reference value”. However, reference population may also be chosen to consist of 20, 30, 50, 200, 500 or 1000 individuals. Healthy individuals represent a preferred reference population for establishing a control value.

For example, a marker concentration in a patient sample can be compared to a concentration known to be associated with a specific course of a certain disease. Usually the sample's marker concentration is directly or indirectly correlated with a diagnosis and the marker concentration is e.g. used to determine whether an individual is at risk for a certain disease. Alternatively, the sample's marker concentration can e.g. be compared to a marker concentration known to be associated with a response to therapy in a certain disease, the diagnosis of a certain disease, the assessment of the severity of a certain disease, the guidance for selecting an appropriate drug to a certain disease, in judging the risk of disease progression, or in the follow-up of patients. Depending on the intended diagnostic use an appropriate control sample is chosen and a control or reference value for the marker established therein. As also clear to the skilled artisan, the absolute marker values established in a control sample will be dependent on the assay used.

The terms “lowered” or “decreased” level of an indicator refer to the level of such indicator in the sample being reduced in comparison to the reference or reference sample.

The terms “elevated” or “increased” level of an indicator refer to the level of such indicator in the sample being higher in comparison to the reference or reference sample. E.g. a protein that is detectable in higher amounts in a fluid sample of one individual suffering from a given disease than in the same fluid sample of individuals not suffering from said disease, has an elevated level.

The term “measurement”, “measuring” or “determining” preferably comprises a qualitative, a semi-quantitative or a quantitative measurement.

The term “immunoglobulin (Ig)” as used herein refers to immunity conferring glycoproteins of the immunoglobulin superfamily. “Surface immunoglobulins” are attached to the membrane of effector cells by their transmembrane region and encompass molecules such as but not limited to B-cell receptors, T-cell receptors, class I and II major histocompatibility complex (MHC) proteins, beta-2 microglobulin (˜2M), CD3, CD4 and CDS.

Typically, the term “antibody” as used herein refers to secreted immunoglobulins which lack the transmembrane region and can thus, be released into the bloodstream and body cavities. Human antibodies are grouped into different isotypes based on the heavy chain they possess. There are five types of human Ig heavy chains denoted by the Greek letters: u, y, 6, E, and p. The type of heavy chain present defines the class of antibody, i.e. these chains are found in IgA, IgD, IgE, IgG, and IgM antibodies, respectively, each performing different roles, and directing the appropriate immune response against different types of antigens. Distinct heavy chains differ in size and composition; and may comprise approximately 450 amino acids (Janeway et al. (2001) Immunobiology, Garland Science). IgA is found in mucosal areas, such as the gut, respiratory tract and urogenital tract, as well as in saliva, tears, and breast milk and prevents colonization by pathogens (Underdown & Schiff (1986) Annu. Rev. Immunol. 4:389-417). IgD mainly functions as an antigen receptor on B cells that have not been exposed to antigens and is involved in activating basophils and mast cells to produce antimicrobial factors (Geisberger et al. (2006) Immunology 118:429-437; Chen et al. (2009) Nat. Immunol. 10:889-898). IgE is involved in allergic reactions via its binding to allergens triggering the release of histamine from mast cells and basophils. IgE is also involved in protecting against parasitic worms (Pier et al. (2004) Immunology, Infection, and Immunity, ASM Press). IgG provides the majority of antibody-based immunity against invading pathogens and is the only antibody isotype capable of crossing the placenta to give passive immunity to fetus (Pier et al. (2004) Immunology, Infection, and Immunity, ASM Press). In humans there are four different IgG subclasses (IgG1, 2, 3, and 4), named in order of their abundance in serum with IgG1 being the most abundant (˜66%), followed by IgG2 (˜23%), IgG3 (˜7%) and IgG (˜4%). The biological profile of the different IgG classes is determined by the structure of the respective hinge region. IgM is expressed on the surface of B cells in a monomeric form and in a secreted pentameric form with very high avidity. IgM is involved in eliminating pathogens in the early stages of B cell mediated (humoral) immunity before sufficient IgG is produced (Geisberger et al. (2006) Immunology 118:429-437). Antibodies are not only found as monomers but are also known to form dimers of two Ig units (e.g. IgA), tetramers of four Ig units (e.g. IgM of teleost fish), or pentamers of five Ig units (e.g. mammalian IgM). Antibodies are typically made of four polypeptide chains comprising two identical heavy chains and identical two light chains which are connected via disulfide bonds and resemble a “Y”-shaped macro-molecule. Each of the chains comprises a number of immunoglobulin domains out of which some are constant domains and others are variable domains. Immunoglobulin domains consist of a 2-layer sandwich of between 7 and 9 antiparallel ˜-strands arranged in two ˜-sheets. Typically, the heavy chain of an antibody comprises four Ig domains with three of them being constant (CH domains: CHI. CH2. CH3) domains and one of the being a variable domain (V H). The light chain typically comprises one constant Ig domain (CL) and one variable Ig domain (V L). Exemplified, the human IgG heavy chain is composed of four Ig domains linked from N- to C-terminus in the order VwCH1-CH2-CH3 (also referred to as VwCy1-Cy2-Cy3), whereas the human IgG light chain is composed of two immunoglobulin domains linked from N- to C-terminus in the order VL-CL, being either of the kappa or lambda type (VK-CK or VA.-CA.). Exemplified, the constant chain of human IgG comprises 447 amino acids.

Throughout the present specification and claims, the numbering of the amino acid positions in an immunoglobulin are that of the “EU index” as in Kabat, E. A., Wu, T. T., Perry, H. M., Gottesman, K. S., and Foeller, C., (1991) Sequences of proteins of immunological interest, 5^{th} ed. U.S. Department of Health and Human Service, National Institutes of Health, Bethesda, Md. The “EU index as in Kabat” refers to the residue numbering of the human IgG IEU antibody. Accordingly, CH domains in the context of IgG are as follows: “CHI” refers to amino acid positions 118-220 according to the EU index as in Kabat; “CH2” refers to amino acid positions 237-340 according to the EU index as in Kabat; and “CH3” refers to amino acid positions 341-44 7 according to the EU index as in Kabat.

The terms “full-length antibody”, “intact antibody”, and “whole antibody” are used herein interchangeably to refer to an antibody in its substantially intact form, not antibody fragments as defined below. The terms particularly refer to an antibody with heavy chains that contain an Fc region.

Papain digestion of antibodies produces two identical antigen binding fragments, called “Fab fragments” (also referred to as “Fab portion” or “Fab region”) each with a single antigen binding site, and a residual “Fe fragment” (also referred to as “Fe portion” or “Fe region”) whose name reflects its ability to crystallize readily. The crystal structure of the human IgG Fe region has been determined (Deisenhofer (1981) Biochemistry 20:2361-2370). In IgG, IgA and IgD isotypes, the Fe region is composed of two identical protein fragments, derived from the CH2 and CH3 domains of the antibody's two heavy chains; in IgM and IgE isotypes, the Fe regions contain three heavy chain constant domains (CH2-4) in each polypeptide chain. In addition, smaller immunoglobulin molecules exist naturally or have been constructed artificially. The term “Fab′ fragment” refers to a Fab fragment additionally comprise the hinge region of an Ig molecule whilst “F(ab′)2 fragments” are understood to comprise two Fab′ fragments being either chemically linked or connected via a disulfide bond. Whilst “single domain antibodies (sdAb)” (Desmyter et al. (1996) Nat. Structure Biol. 3:803-811) and “Nanobodies” only comprise a single VH domain, “single chain Fv (scFv)” fragments comprise the heavy chain variable domain joined via a short linker peptide to the light chain variable domain (Huston et al. (1988) Proc. Natl. Acad. Sci. USA 85, 5879-5883). Divalent single-chain variable fragments (di-scFvs) can be engineered by linking two scFvs (scFvA-scFvB). This can be done by producing a single peptide chain with two VH and two VL regions, yielding “tandem scFvs” (VHA-VLA-VHB-VLB). Another possibility is the creation of scFvs with linkers that are too short for the two variable regions to fold together, forcing scFvs to dimerize. Usually linkers with a length of 5 residues are used to generate these dimers. This type is known as “diabodies”. Still shorter linkers (one or two amino acids) between a V H and V L domain lead to the formation of monospecific trimers, so-called “triabodies” or “tribadies”. Bispecific diabodies are formed by expressing to chains with the arrangement VHA-VLB and VHB-VLA or VLA-VHB and VLB-VHA, respectively. Singlechain diabodies (scDb) comprise a VHA-VLB and a VHB-VLA fragment which are linked by a linker peptide (P) of 12-20 amino acids, preferably 14 amino acids, (VHA-VLB-P-VHB-VLA). “Bi-specific T-cell engagers (BiTEs)” are fusion proteins consisting of two scFvs of different antibodies wherein one of the scFvs binds to T cells via the CD3 receptor, and the other to a tumor cell via a tumor specific molecule (Kufer et al. (2004) Trends Biotechnol. 22:238-244). Dual affinity retargeting molecules (“DART” molecules) are diabodies additionally stabilized through a C-terminal disulfide bridge.

Accordingly, the term “antibody fragments” refers to a portion of an intact antibody, preferably comprising the antigen-binding region thereof. Antibody fragments include but are not limited to Fab, Fab′, F(ab′)2, Fv fragments; diabodies; sdAb, nanobodies, scFv, di-scFvs, tandem scFvs, triabodies, diabodies, scDb, BiTEs, and DARTs.

The term “binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including but not limited to surface plasmon resonance based assay (such as the BIAcore assay as described in PCT Application Publication No. WO2005/012359); enzyme-linked immunoabsorbent assay (ELISA); and competition assays (e.g. RIA's). Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention.

“Sandwich immunoassays” are broadly used in the detection of an analyte of interest. In such assay the analyte is “sandwiched” in between a first antibody and a second antibody. Typically, a sandwich assay requires that capture and detection antibody bind to different, non-overlapping epitopes on an analyte of interest. By appropriate means such sandwich complex is measured and the analyte thereby quantified. In a typical sandwich-type assay, a first antibody bound to the solid phase or capable of binding thereto and a detectably-labeled second antibody each bind to the analyte at different and non-overlapping epitopes. The first analyte-specific binding agent (e.g. an antibody) is either covalently or passively bound to a solid surface. The solid surface is typically glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g., from room temperature to 40° C. such as between 25° C. and 37° C. inclusive) to allow for binding between the first or capture antibody and the corresponding antigen. Following the incubation period, the solid phase, comprising the first or capture antibody and bound thereto the antigen can be washed, and incubated with a secondary or labeled antibody binding to another epitope on the antigen. The second antibody is linked to a reporter molecule which is used to indicate the binding of the second antibody to the complex of first antibody and the antigen of interest.

An extremely versatile alternative sandwich assay format includes the use of a solid phase coated with the first partner of a binding pair, e.g. paramagnetic streptavidin-coated microparticles. Such microparticles are mixed and incubated with an analyte-specific binding agent bound to the second partner of the binding pair (e.g. a biotinylated antibody), a sample suspected of comprising or comprising the analyte, wherein said second partner of the binding pair is bound to said analyte-specific binding agent, and a second analyte-specific binding agent which is detectably labeled. As obvious to the skilled person these components are incubated under appropriate conditions and for a period of time sufficient for binding the labeled antibody via the analyte, the analyte-specific binding agent (bound to) the second partner of the binding pair and the first partner of the binding pair to the solid phase microparticles. As appropriate such assay may include one or more washing step(s).

The term “detectably labeled” encompasses labels that can be directly or indirectly detected.

Directly detectable labels either provide a detectable signal or they interact with a second label to modify the detectable signal provided by the first or second label, e.g. to give FRET (fluorescence resonance energy transfer). Labels such as fluorescent dyes and luminescent (including chemiluminescent and electrochemiluminescent) dyes (Briggs et al “Synthesis of Functionalised Fluorescent Dyes and Their Coupling to Amines and Amino Acids,” J. Chem. Soc., Perkin-Trans. 1 (1997) 1051-1058) provide a detectable signal and are generally applicable for labeling. In one embodiment detectably labeled refers to a label providing or inducible to provide a detectable signal, i.e. to a fluorescent label, to a luminescent label (e.g. a chemiluminescent label or an electrochemiluminescent label), a radioactive label or a metal-chelate based label, respectively.

Numerous labels (also referred to as dyes) are available which can be generally grouped into the following categories, all of them together and each of them representing embodiments according the present disclosure:

(a) Fluorescent dyes

Fluorescent dyes are e.g. described by Briggs et al “Synthesis of Functionalized Fluorescent Dyes and Their Coupling to Amines and Amino Acids,” J. Chem. Soc., Perkin-Trans. 1 (1997) 1051-1058).

Fluorescent labels or fluorophores include rare earth chelates (europium chelates), fluorescein type labels including FITC, 5-carboxyfluorescein, 6-carboxy fluorescein; rhodamine type labels including TAMRA; dansyl; Lissamine; cyanines; phycoerythrins; Texas Red; and analogs thereof. The fluorescent labels can be conjugated to an aldehyde group comprised in target molecule using the techniques disclosed herein. Fluorescent dyes and fluorescent label reagents include those which are commercially available from Invitrogen/Molecular Probes (Eugene, Oreg., USA) and Pierce Biotechnology, Inc. (Rockford, Ill.).

(b) Luminescent dyes

Luminescent dyes or labels can be further subcategorized into chemiluminescent and electrochemiluminescent dyes.

The different classes of chemiluminogenic labels include luminol, acridinium compounds, coelenterazine and analogues, dioxetanes, systems based on peroxyoxalic acid and their derivatives. For immunodiagnostic procedures predominantly acridinium based labels are used (a detailed overview is given in Dodeigne C. et al., Talanta 51 (2000) 415-439).

The labels of major relevance used as electrochemiluminescent labels are the Ruthenium- and the Iridium-based electrochemiluminescent complexes, respectively. Electrochemiluminescense (ECL) proved to be very useful in analytical applications as a highly sensitive and selective method. It combines analytical advantages of chemiluminescent analysis (absence of background optical signal) with ease of reaction control by applying electrode potential. In general Ruthenium complexes, especially [Ru(Bpy)3]2+(which releases a photon at ˜620 nm) regenerating with TPA (Tripropylamine) in liquid phase or liquid-solid interface are used as ECL-labels.

Electrochemiluminescent (ECL) assays provide a sensitive and precise measurement of the presence and concentration of an analyte of interest. Such techniques use labels or other reactants that can be induced to luminesce when electrochemically oxidized or reduced in an appropriate chemical environment. Such electrochemiluminescense is triggered by a voltage imposed on a working electrode at a particular time and in a particular manner. The light produced by the label is measured and indicates the presence or quantity of the analyte. For a fuller description of such ECL techniques, reference is made to U.S. Pat. Nos. 5,221,605, 5,591,581, 5,597,910, PCT published application WO90/05296, PCT published application WO92/14139, PCT published application WO90/05301, PCT published application WO96/24690, PCT published application US95/03190, PCT application US97/16942, PCT published application US96/06763, PCT published application WO95/08644, PCT published application WO96/06946, PCT published application WO96/33411, PCT published application WO87/06706, PCT published application WO96/39534, PCT published application WO96/41175, PCT published application WO96/40978, PCT/US97/03653 and U.S. patent application Ser. No. 08/437,348 (U.S. Pat. No. 5,679,519). Reference is also made to a 1994 review of the analytical applications of ECL by Knight, et al. (Analyst, 1994, 119: 879-890) and the references cited therein. In one embodiment the method according to the present description is practiced using an electrochemiluminescent label.

Recently also Iridium-based ECL-labels have been described (WO2012107419).

(c) Radioactive labels make use of radioisotopes (radionuclides), such as 3H, 1 IC, 14C, 18F, 32P, 35S, 64Cu, 68Gn, 86Y, 89Zr, 99TC, 111In, 123I, 124I, 125I, 131I, 133Xe, 177Lu, 211At, or 131Bi.

(d) Metal-chelate complexes suitable as labels for imaging and therapeutic purposes are well-known in the art (US 2010/0111861; U.S. Pat. Nos. 5,342,606; 5,428,155; 5,316,757; 5,480,990; 5,462,725; 5,428,139; 5,385,893; 5,739,294; 5,750,660; 5,834,461; Hnatowich et al, J. Immunol. Methods 65 (1983) 147-157; Meares et al, Anal. Biochem. 142 (1984) 68-78; Mirzadeh et al, Bioconjugate Chem. 1 (1990) 59-65; Meares et al, J. Cancer (1990), Suppl. 10:21-26; Izard et al, Bioconjugate Chem. 3 (1992) 346-350; Nikula et al, Nucl. Med. Biol. 22 (1995) 387-90; Camera et al, Nucl. Med. Biol. 20 (1993) 955-62; Kukis et al, J. Nucl. Med. 39 (1998) 2105-2110; Verel et al., J. Nucl. Med. 44 (2003) 1663-1670; Camera et al, J. Nucl. Med. 21 (1994) 640-646; Ruegg et al, Cancer Res. 50 (1990) 4221-4226; Verel et al, J. Nucl. Med. 44 (2003) 1663-1670; Lee et al, Cancer Res. 61 (2001) 4474-4482; Mitchell, et al, J. Nucl. Med. 44 (2003) 1105-1112; Kobayashi et al Bioconjugate Chem. 10 (1999) 103-111; Miederer et al, J. Nucl. Med. 45 (2004) 129-137; DeNardo et al, Clinical Cancer Research 4 (1998) 2483-90; Blend et al, Cancer Biotherapy & Radiopharmaceuticals 18 (2003) 355-363; Nikula et al J. Nucl. Med. 40 (1999) 166-76; Kobayashi et al, J. Nucl. Med. 39 (1998) 829-36; Mardirossian et al, Nucl. Med. Biol. 20 (1993) 65-74; Roselli et al, Cancer Biotherapy & Radiopharmaceuticals, 14 (1999) 209-20).

### EMBODIMENTS

In a first aspect, the present invention relates to a method of assessing whether a patient has endometriosis or is at risk of developing endometriosis, comprising

a) determining the amount of S100A12 in a sample of the patient, and

b) comparing the determined amount to a reference.

In embodiments, an elevated amount of S100A12 in the sample of the patient is indicative of the presence or the risk of developing endometriosis in the patient. In particular, an amount of S100A12 in the sample of the patient is indicative of the presence or the risk of developing of endometriosis in the patient if the amount of S100A12 in the sample of the patient is higher than the amount of S100A12 in a reference or reference sample. In particular, S100A12 is detectable in higher amounts in a fluid sample of the patient assessed for the presence or risk of developing endometriosis than in the same fluid sample of individuals not suffering or being at risk of developing endometriosis.

In particular, an amount of S100A12 elevated by 50% or more, is indicative of the presence or the risk of developing of endometrioses. In particular, an amount of S100A12 elevated by 100% or more, is indicative of the presence or the risk of developing of endometrioses. In particular, an amount of S100A12 elevated by 150% or more, is indicative of the presence or the risk of developing of endometrioses. In particular, an amount of S100A12 elevated by 200% or more, is indicative of the presence or the risk of developing of endometrioses.

In embodiments, the sample of the patient is body fluid sample. In particular embodiments, the sample is a whole blood, serum or plasma sample. In embodiments, the sample is an in vitro sample, i.e. it will be analyzed in vitro and not transferred back into the body.

In particular embodiments, the patient is a laboratory animal, a domestic animal or a primate. In particular embodiments, the patient is a human patient. In particular embodiments, the patient is a female human patient.

In embodiments, the endometriosis assessed is selected from the group consisting of stage I endometriosis according to rASRM staging, stage II endometriosis according to rASRM staging, stage III endometriosis according to rASRM staging, stage IV endometriosis according to rASRM staging. In particular embodiments, the endometriosis assessed is stage I, stage II, stage III, or stage IV endometriosis. In embodiments, endometriosis is early endometriosis, in particular stage I endometriosis according to rASRM staging or stage II endometriosis according to rASRM staging.

In particular embodiments, the endometriosis assessed is stage III or stage IV endometriosis.

In embodiments, the endometriosis assessed is selected from the group consisting of peritoneal endometriosis, endometrioma, deep infiltrating endometriosis (DIE), and adenomyosis.

In particular embodiments, the endometriosis assessed is peritoneal endometriosis of stage I or II according to rASRM staging.

In embodiments, the assessment is performed independent of the rASRM staging. In particular, the assessment is performed without performing laparoscopy. In particular the assessment is performed without assessing the presence or severity of endometriosis in the patient using laparoscopy and/or the rASRM staging.

In embodiment, the method of the present invention is an in vitro method.

In embodiments, the amount of S100A12 is determined using antibodies, in particular using monoclonal antibodies. In embodiments, step a) of determining the amount of S100A12 in a sample of the patient comprises performing an immunoassay. In embodiments, the immunoassay is performed either in a direct or indirect format. In embodiments such immunoassays is selected from the group consisting of enzyme linked immunosorbent assay (ELISA), enzyme immunoassay (EIA), radioimmunoassay (RIA), or immuno assays based on detection of luminescence, fluorescence, chemiluminescence or electrochemiluminescence.

In particular embodiments, step a) of determining the amount of S100A12 in a sample of the patient comprises the steps of
i) incubating the sample of the patient with one or more antibodies specifically binding to S100A12, thereby generating a complex between the antibody and S100A12, and
ii) quantifying the complex formed in step i), thereby quantifying the amount of S100A12 in the sample of the patient.

In particular embodiments, in step i) the sample is incubated with two antibodies, specifically binding to S100A12. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first anti-S100A12 antibody/S100A12/second anti-S100A12 antibody complex. As the skilled artisan will readily appreciate it is nothing but routine experimentation to establish the time and conditions that are appropriate or that are sufficient for the formation of a complex either between the specific anti-S100A12 antibody and the S100A12 antigen/analyte (=anti-S100A12 complex) or the formation of the secondary, or sandwich complex comprising the first antibody to S100A12, S100A12 (the analyte) and the second anti-S100A12 antibody (=anti-S100A12 antibody/S100A12/second anti-S100A12 antibody complex).

The detection of the anti-S100A12 antibody/S100A12 complex can be performed by any appropriate means. The detection of the first anti-S100A12 antibody/S100A12/second anti-S100A12 antibody complex can be performed by any appropriate means. The person skilled in the art is absolutely familiar with such means/methods.

In certain embodiments a sandwich will be formed comprising a first antibody to S100A12, S100A12 (analyte) and the second antibody to S100A12, wherein the second antibody is detectably labeled.

In one embodiment a sandwich will be formed comprising a first antibody to S100A12, the S100A12 (analyte) and the second antibody to S100A12, wherein the second antibody is detectably labeled and wherein the first anti-S100A12 antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

In embodiments, the method further comprising the assessment of the presence of dysmenorrhea and/or lower abdominal pain in the patient. In embodiments the presence of dysmenorrhea and/or lower abdominal pain is assessed according to the VAS scale. In embodiments, dysmenorrhea VAS score of 4 or higher indicated moderate or severe dysmenorrhea. In embodiments, scores of 3 or less indicate no or mild dysmenorrhea. In embodiments, the method further comprising determining the amount or concentration of CA-125.

In embodiments, the method comprising calculating a ratio of the amount or concentration of S100A12 and dysmenorrhea, of the amount or concentration of S100A12 and lower abdominal pain according to the VAS scale, or the amount or concentration of S100A12 and the amount or concentration of CA-125.

In a second aspect, the present invention relates to a method of selecting a patient for therapy of endometriosis, comprising
a) determining the amount or concentration of S100A12 in a sample of the patient, and
b) comparing the determined amount or concentration to a reference.

In embodiments, a patient is selected for therapy of endometriosis if an elevated amount of S100A12 in the sample of the patient is determined. In particular, a patient is selected for therapy of endometriosis if the amount of S100A12 in the sample of the patient is higher than the amount of S100A12 in a reference or reference sample. In particular, a patient is selected for therapy of endometriosis if the amount of S100A12 is higher in a fluid sample of the patient than in the same fluid sample of individuals not suffering or being at risk of developing endometriosis or not being selected for therapy of endometriosis.

In particular, a patient is selected for therapy of endometriosis if the amount of S100A12 is elevated by 50% or more. In particular, a patient is selected for therapy of endometriosis if the amount of S100A12 is elevated by 100% or more. In particular, a patient is selected for therapy of endometriosis if the amount of S100A12 is elevated by 150% or more. In particular, a patient is selected for therapy of endometriosis if the amount of S100A12 is elevated by 200% or more.

In embodiments, the patient is selected for a therapy of endometriosis selected from the group consisting of drug-based therapy or surgical therapy. In embodiments surgical therapy of endometriosis is laparoscopy or nerve sparing surgery. In embodiments drug-based therapy of endometriosis is inhibiting or targeting neurogenic inflammation and/or pain medication and/or hormonal therapy (e.g. hormonal contraceptives or GnRH agonists).

In embodiments, the sample of the patient is body fluid sample. In particular embodiments, the sample is a whole blood, serum or plasma sample. In embodiments, the sample is an in vitro sample, i.e. it will be analyzed in vitro and not transferred back into the body.

In particular embodiments, the patient is a laboratory animal, a domestic animal or a primate. In particular embodiments, the patient is a human patient. In particular embodiments, the patient is a female human patient.

In embodiments, the endometriosis is selected from the group consisting of stage I endometriosis according to rASRM staging, stage II endometriosis according to rASRM staging, stage III endometriosis according to rASRM staging, stage IV endometriosis according to rASRM staging. In particular embodiments, the endometriosis is stage I, stage II, stage III, or stage IV endometriosis. In embodiments, endometriosis is early endometriosis, in particular stage I endometriosis according to rASRM staging or stage II endometriosis according to rASRM staging. In particular embodiments, the endometriosis assessed is stage III or stage IV endometriosis.

In embodiments, the endometriosis is selected from the group consisting of peritoneal endometriosis, endometrioma, deep infiltrating endometriosis (DIE), and adenomyosis.

In particular embodiments, the endometriosis assessed is peritoneal endometriosis of stage I or II according to rASRM staging.

In embodiment, the method of the present invention is an in vitro method.

In embodiments, the amount of S100A12 is determined using antibodies, in particular using monoclonal antibodies. In embodiments, step a) of determining the amount of S100A12 in a sample of the patient comprises performing an immunoassay. In embodiments, the immunoassay is performed either in a direct or indirect format. In embodiments such immunoassays is selected from the group consisting of enzyme linked immunosorbent assay (ELISA), enzyme immunoassay (EIA), radioimmunoassay (RIA), or immuno assays based on detection of luminescence, fluorescence, chemiluminescence or electrochemiluminescence.

In particular embodiments, step a) of determining the amount of S100A12 in a sample of the patient comprises the steps of
i) incubating the sample of the patient with one or more antibodies specifically binding to S100A12, thereby generating a complex between the antibody and S100A12, and
ii) quantifying the complex formed in step i), thereby quantifying the amount of S100A12 in the sample of the patient.

In particular embodiments, in step i) the sample is incubated with two antibodies, specifically binding to S100A12. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first anti-S100A12 antibody/S100A12/second anti-S100A12 antibody complex. As the skilled artisan will readily appreciate it is nothing but routine experimentation to establish the time and conditions that are appropriate or that are sufficient for the formation of a complex either between the specific anti-S100A12 antibody and the S100A12 antigen/analyte (=anti-S100A12 complex) or the formation of the secondary, or sandwich complex comprising the first antibody to S100A12, S100A12 (the analyte) and the second anti-S100A12 antibody (=anti-S100A12 antibody/S100A12/second anti-S100A12 antibody complex).

The detection of the anti-S100A12 antibody/S100A12 complex can be performed by any appropriate means. The detection of the first anti-S100A12 antibody/S100A12/second anti-S100A12 antibody complex can be performed by any appropriate means. The person skilled in the art is absolutely familiar with such means/methods.

In certain embodiments, a sandwich will be formed comprising a first antibody to S100A12, S100A12 (analyte) and the second antibody to S100A12, wherein the second antibody is detectably labeled.

In one embodiment a sandwich will be formed comprising a first antibody to S100A12, the S100A12 (analyte) and the second antibody to S100A12, wherein the second antibody is detectably labeled and wherein the first anti-S100A12 antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

In embodiments, the method further comprising the assessment of the presence of dysmenorrhea and/or lower abdominal pain in the patient. In embodiments the presence of dysmenorrhea and/or lower abdominal pain is assessed according to the VAS scale. In embodiments, dysmenorrhea VAS score of 4 or higher indicated moderate or severe dysmenorrhea. In embodiments, scores of 3 or less indicate no or mild dysmenorrhea.

In embodiments, the method further comprising determining the amount or concentration of CA-125.

In embodiments, the method comprising calculating a ratio of the amount or concentration of S100A12 and dysmenorrhea, of the amount or concentration of S100A12 and lower abdominal pain according to the VAS scale, or the amount or concentration of S100A12 and the amount or concentration of CA-125.

In a third aspect, the present invention relates to a method of monitoring a patient suffering from endometriosis or being treated for endometriosis, comprising
a) determining the amount or concentration of S100A12 in a sample of the patient, and
b) comparing the determined amount or concentration to a reference.

In embodiments, a patient suffering from endometriosis is monitored to determine if the amount or concentration of S100A12 is changing over time in a sample of the patient. In particular, a patient suffering from endometriosis is monitored to determine if the amount or concentration of S100A12 is increasing, decreasing or not changing over time. In embodiments, a patient suffering from endometriosis is monitored if an elevated amount of S100A12 in the sample of the patient is determined.

In embodiments, a patient being treated for endometriosis is monitored to determine if the amount or concentration of S100A12 is changing in a sample of the patient. In particular, a patient being treated for endometriosis is monitored to determine if the amount or concentration of S100A12 is increasing, decreasing or not changing. In particular, a patient being treated for endometriosis is monitored to determine if the amount or concentration of S100A12 is increasing, decreasing or not changing due to the therapy applied. In embodiments, a decreasing amount or concentration of S100A12 in a patient being treated for endometriosis is indicative of the therapy being effective. In embodiments, an unaltered or increasing amount or concentration of S100A12 in a sample of the patient being treated for endometriosis is indicative of the therapy being ineffective, i.e. an unaltered or increasing amount or concentration of S100A12 in a sample of the patient being treated for endometriosis is indicative of persisting or recurring endometriosis. In particular, the treatment for endometriosis is ineffective if the amount of S100A12 is increasing to 50% or more. In particular, the treatment for endometriosis is ineffective if the amount of S100A12 is increasing to 100% or more. In particular, the treatment for endometriosis is ineffective if the amount of S100A12 is increasing to 150% or more. In particular, the treatment for endometriosis is ineffective if the amount of S100A12 is increasing to 200% or more.

In particular embodiments, therapy is adapted if an unaltered or increasing amount or concentration of S100A12 in a sample of the patient being treated for endometriosis is determined.

In embodiments, the patient is monitored several times at different time points. In embodiments, the patient is monitored several times within a time frame of weeks, months or years. In particular embodiments, a patient is monitored is once a months or once a year. In embodiments, a patient suffering from endometriosis is monitored once a months or once a year after diagnosis of endometriosis. In embodiments, a patient being treated for endometriosis is monitored once after therapy, in particular once after surgical therapy. In particular, the patient being treated for endometriosis is monitored once a months or once a year to determine the efficacy of treatment and/or the recurrence of endometriosis.

In embodiments, therapy of endometriosis is selected from the group consisting of drug-based therapy or surgical therapy. In embodiments, surgical therapy of endometriosis is laparoscopy or nerve sparing surgery. In embodiments, drug-based therapy of endometriosis is inhibiting or targeting neurogenic inflammation and/or pain medication and/or hormonal therapy. In embodiments, the sample of the patient is body fluid sample. In particular embodiments, the sample is a whole blood, serum or plasma sample. In embodiments, the sample is an in vitro sample, i.e. it will be analyzed in vitro and not transferred back into the body.

In particular embodiments, the patient is a laboratory animal, a domestic animal or a primate. In particular embodiments, the patient is a human patient. In particular embodiments, the patient is a female human patient.

In embodiments, the endometriosis is selected from the group consisting of stage I endometriosis according to rASRM staging, stage II endometriosis according to rASRM staging, stage III endometriosis according to rASRM staging, stage IV endometriosis according to rASRM staging. In particular embodiments, the endometriosis is stage I, stage II, stage III, or stage IV endometriosis. In embodiments, endometriosis is early endometriosis, in particular stage I endometriosis according to rASRM staging or stage II endometriosis according to rASRM staging. In particular embodiments, the endometriosis assessed is stage III or stage IV endometriosis.

In embodiments, the endometriosis is selected from the group consisting of peritoneal endometriosis, endometrioma, deep infiltrating endometriosis (DIE), and adenomyosis.

In particular embodiments, the endometriosis assessed is peritoneal endometriosis of stage I or II according to rASRM staging.

In embodiment, the method of the present invention is an in vitro method.

In embodiments, the amount of S100A12 is determined using antibodies, in particular using monoclonal antibodies. In embodiments, step a) of determining the amount of S100A12 in a sample of the patient comprises performing an immunoassay. In embodiments, the immunoassay is performed either in a direct or indirect format. In embodiments such immunoassays is selected from the group consisting of enzyme linked immunosorbent assay (ELISA), enzyme immunoassay (EIA), radioimmunoassay (RIA), or immuno assays based on detection of luminescence, fluorescence, chemiluminescence or electrochemiluminescence.

In particular embodiments, step a) of determining the amount of S100A12 in a sample of the patient comprises the steps of
i) incubating the sample of the patient with one or more antibodies specifically binding to S100A12, thereby generating a complex between the antibody and S100A12, and
ii) quantifying the complex formed in step i), thereby quantifying the amount of S100A12 in the sample of the patient.

In particular embodiments, in step i) the sample is incubated with two antibodies, specifically binding to S100A12. As obvious to the skilled artisan, the sample can be contacted with the first and the second antibody in any desired order, i.e. first antibody first and then the second antibody or second antibody first and then the first antibody, or simultaneously, for a time and under conditions sufficient to form a first anti-S100A12 antibody/S100A12/second anti-S100A12 antibody complex. As the skilled artisan will readily appreciate it is nothing but routine experimentation to establish the time and conditions that are appropriate or that are sufficient for the formation of a complex either between the specific anti-S100A12 antibody and the S100A12 antigen/analyte (=anti-S100A12 complex) or the formation of the secondary, or sandwich complex comprising the first antibody to S100A12, S100A12 (the analyte) and the second anti-S100A12 antibody (=anti-S100A12 antibody/S100A12/second anti-S100A12 antibody complex).

The detection of the anti-S100A12 antibody/S100A12 complex can be performed by any appropriate means. The detection of the first anti-S100A12 antibody/S100A12/second anti-S100A12 antibody complex can be performed by any appropriate means. The person skilled in the art is absolutely familiar with such means/methods.

In certain embodiments, a sandwich will be formed comprising a first antibody to S100A12, S100A12 (analyte) and the second antibody to S100A12, wherein the second antibody is detectably labeled.

In one embodiment a sandwich will be formed comprising a first antibody to S100A12, the S100A12 (analyte) and the second antibody to S100A12, wherein the second antibody is detectably labeled and wherein the first anti-S100A12 antibody is capable of binding to a solid phase or is bound to a solid phase.

In embodiments, the second antibody is directly or indirectly detectably labeled. In particular embodiments, the second antibody is detectably labeled with a luminescent dye, in particular a chemiluminescent dye or an electrochemiluminescent dye.

In embodiments, the method further comprising the assessment of the presence of dysmenorrhea and/or lower abdominal pain in the patient. In embodiments the presence of dysmenorrhea and/or lower abdominal pain is assessed according to the VAS scale. In embodiments, dysmenorrhea VAS score of 4 or higher indicated moderate or severe dysmenorrhea. In embodiments, scores of 3 or less indicate no or mild dysmenorrhea.

In embodiments, the method further comprising determining the amount or concentration of CA-125.

In embodiments, the method comprising calculating a ratio of the amount or concentration of S100A12 and dysmenorrhea, of the amount or concentration of S100A12 and lower abdominal pain according to the VAS scale, or the amount or concentration of S100A12 and the amount or concentration of CA-125.

In further embodiments, the present invention relates to the following aspects:
1. Method of assessing whether a patient has endometriosis or is at risk of developing endometriosis, comprising
   determining the amount or concentration of S100A12 in a sample of the patient, and
   comparing the determined amount or concentration to a reference.
2. Method of selecting a patient for therapy (in particular drug-based therapy or surgical therapy (laparoscopy) of endometriosis, comprising
   determining the amount or concentration of S100A12 in a sample of the patient, and
   comparing the determined amount or concentration to a reference.
3. Method of monitoring a patient suffering from endometriosis or being treated for endometriosis, comprising
   determining the amount or concentration of S100A12 in a sample of the patient, and
   comparing the determined amount or concentration to a reference.
4. The method of aspects 1 to 3, wherein an elevated amount or concentration of S100A12 in the sample of the patient is indicative of the presence of endometriosis in the patient.
5. The method of aspects 1 to 4, wherein the sample is body fluid.
6. The method of aspects 1 to 5, wherein the sample is blood, serum or plasma.
7. The method of aspects 1 to 6, wherein the subject is a female patient, in particular a human female patient.
8. The method of aspects 1 to 7, wherein the assessment is performed independent of the rASRM staging.
9. The method of aspects 1 to 8, wherein endometriosis is selected from the group consisting of stage I endometriosis according to rASRM staging, stage II endometriosis according to rASRM staging, stage III endometriosis according to rASRM staging, stage IV endometriosis according to rASRM staging.
10. The method of aspects 1 to 9, wherein endometriosis is early endometriosis, in particular stage I endometriosis according to rASRM staging or stage II endometriosis according to rASRM staging.
11. The method of aspects 1 to 10, wherein endometriosis is selected from the group consisting of peritoneal endometriosis, endometrioma, deep infiltrating endometriosis, and adenomyosis.
12. The method of aspects 1 to 11, further comprising the assessment of dysmenorrhea according to the VAS scale and/or lower abdominal pain according to the VAS scale.
13. The method of aspects 1 to 12, further comprising determining the amount or concentration of CA-125.
14. The method of aspects 12 or 13, comprising calculating a ratio of the amount or concentration of S100A12 and dysmenorrhea, of the amount or concentration of S100A12 and lower abdominal pain according to the VAS scale, or the amount or concentration of S100A12 and the amount or concentration of CA-125.

The following examples and figures are provided to aid the understanding of the present invention, the true scope of which is set forth in the appended claims. It is understood that modifications can be made in the procedures set forth without departing from the spirit of the invention.

### Examples

### Example 1: Diagnostic Performance of Biomarker S100A12 and Biomarker Combinations in Women with Endometriosis and Controls

For the measurements, a total of 21 serum as well as 31 plasma samples from human females were analysed. The concentration of the analytes was determined by ELISA (enzyme-linked immunosorbent assay). The case group is comprised of patients diagnosed with pelvic endometriosis (rASRM stages I-IV) diagnosed by laparoscopic visualization with subsequent histological confirmation and the control group including healthy women without endometriosis.

The concentration of S100A12 in human serum was determined using the Human S100A12/EN-RAGE ELISA kit Ver.2 from CircuLex/MBL (distributed by Biozol Eching, Germany; catalogue number: CY-8058V2). The kit utilizes the quantitative sandwich ELISA technique. Microtiter plates are pre-coated with a monoclonal antibody specific for human S100A12. Samples are measured in 200-fold dilution. After bringing all reagents to room temperature 100 μL of each sample and standard are added. Samples are measured in singlicates, standards in duplicates. During 1 h incubation at room temperature on a microplate shaker set to 650 rpm, any S100A12 present is bound to the immobilized capture antibody on the microtiter plate. During washing step (4×350 μL), unbound substances are removed from the plate before 100 μL of an enzyme-linked monoclonal antibody specific for S100A12 is added to the wells. Following 1 h incubation on a shaker and another washing step to remove any unbound detection antibody, 100 μL of substrate solution is added to the plate. Within the next 10 min, the color develops in proportion to the amount of EN-RAGE bound in the initial step. Color development is stopped by addition of 50 μL stop solution and colour intensity is measured with a plate reader at 450 nm for detection and 570 nm for background subtraction. For generation of calibrator curves, lyophilized, recombinant S100A12 delivered with the kit was reconstituted and diluted in calibrator diluent. The calibration range of the assay is 20 pg/mL to 640 pg/mL. Calibrator 6 (640 pg/mL) is prepared by 20-fold dilution of stock solution in calibrator diluent and calibrator 5 to calibrator 1 (20 pg/mL) are prepared by serial 2-fold dilution steps in calibrator diluent. Pure calibrator diluent serves as blank (0 pg/mL). The calibration curves were fitted using a 4-parameter nonlinear regression (Newton/Raphson) with no weighting.

The concentration of CA-125 was determined by a cobas e 601 analyzer. Detection of CA 125 II with a cobas e 601 analyzer is based on the Elecsys® Electro-ChemiLuminescence (ECL) technology. In brief, biotin-labelled and ruthenium-labelled antibodies are combined with the respective amount of undiluted sample and incubated on the analyzer. Subsequently, streptavidin-coated magnetic microparticles are added and incubated on the instrument in order to facilitate binding of the biotin-labelled immunological complexes. After this incubation step the reaction mixture is transferred into the measuring cell where the beads are magnetically captured on the surface of an electrode. ProCell M Buffer containing tripropylamine (TPA) for the subsequent ECL reaction is then introduced into the measuring cell in order to separate bound immunoassay complexes from the free remaining particles. Induction of voltage between the working and the counter electrode then initiates the reaction leading to emission of photons by the ruthenium complexes as well as TPA. The resulting electrochemiluminescent signal is recorded by a photomultiplier and converted into numeric values indicating concentration level of the respective analyte.

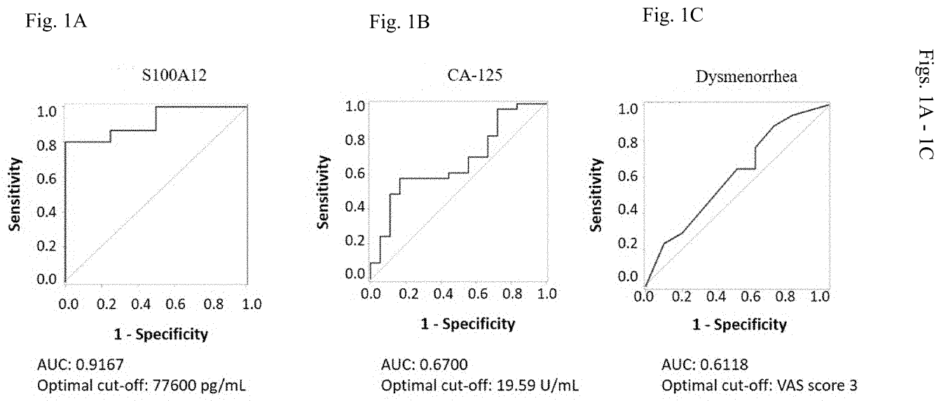

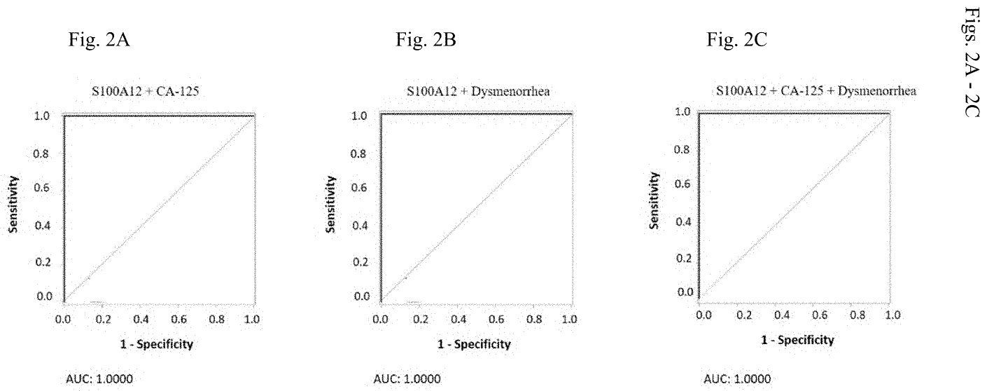

Receiver Operating Characteristic (ROC) curves were generated (see FIGS. 1A-1C for single biomarkers and FIGS. 2A-2C for biomarker combinations). The model performance is determined by looking at the area under the curve (AUC). The best possible AUC is 1 while the lowest possible is 0.5.

TABLE 1
Diagnostic performance of biomarker S100A12 and biomarker
combinations in women with endometriosis and controls

| | | 95% CI | N | |
|---|---|---|---|---|
| | | (confidence | (sample | |
| Marker | AUC | interval) | size) | Sample type |
| S100A12 | 0.9167 | 0.7825 | 19 | Serum |
| (ENRAGE) | | 1.0000 | | |
| CA-125 | 0.6700 | 0.5136 | 51 | Serum/Plasma |
| | | 0.8264 | | |
| Dysmenorrhea | 0.6118 | 0.3829 | 27 | Serum/Plasma |
| | | 0.8406 | | |
| S100A12 | 1.0000 | 1.0000 | 17 | Serum |
| (ENRAGE) + | | 1.0000 | | |
| CA-125 | | | | |
| S100A12 | 1.0000 | 1.0000 | 10 | Serum |
| (ENRAGE) + | | 1.0000 | | |
| Dysmenorrhea | | | | |
| S100A12 | 1.0000 | 1.0000 | 10 | Serum |
| (ENRAGE) + | | 1.0000 | | |
| CA-125 + | | | | |
| Dysmenorrhea | | | | |

For the multivariate analysis, AUC plots, a cut-off for the combination of the different biomarker was applied to predict endometriosis based on multivariable logistic regression analysis and Youden's index. A dysmenorrhea VAS score of 4 or higher indicated moderate or severe dysmenorrhea. Scores of 3 or less indicate no or mild dysmenorrhea.

Multivariable logistic regression model with S100A12 and CA-125:
If logit=α+(β1*S100A12 value [pg/mL])+(β2*CA-125 value [U/mL])≥cut-off then disease (i.e. Endometriosis Stage I, II, III or IV) else no disease

or
If logit=−43.0166+(0.000487*S100A12)+(1.1660*CA-125 value)≥3.717396 then disease (i.e. Endometriosis Stage I, II, III or IV), else no disease

TABLE 2
Cut-off for combination of various biomarkers to
predict endometriosis based on multivariable logistic
regression analysis and Youden's index

| | | | Dysmenorrhea | Cut-off |
|---|---|---|---|---|
| Intercept | S100A12 | CA-125 | VAS | (logit) for |
| α* | β₁** | β₂** | β₃** | Endometriosis |
| −22.2958 | 0.000321 | — | 20.4128 | ≥8.659907 |
| −43.0166 | 0.000487 | 1.1660 | — | ≥3.717396 |
| −20.2648 | 0.000329 | −0.2120 | 35.1227 | ≥6.826602 |

*Intercept α: The point where the curve crosses the y axis;
**Parameter β: The slope of the linear regression curve for each variable.

The values of parameters α and β vary depending on the analytes and clinical symptom included in the multivariate analysis, respectively.

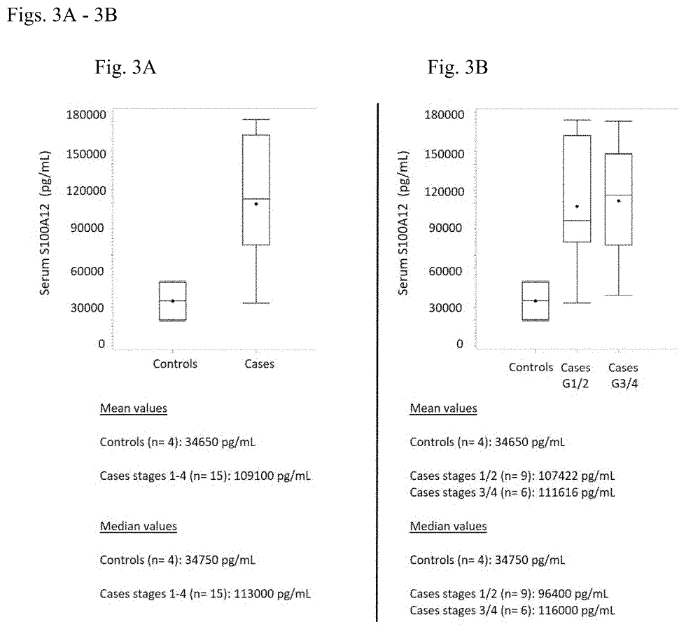

Box plots (see FIGS. 3A & 3B) were generated for endometriosis cases versus controls (FIG. 3A) and endometriosis cases G1/2 (rASRM Stage I-II) versus endometriosis cases G3-4 (rASRM Stage III-IV)/controls (FIG. 3B). The data are presented using box and whisker plots, including the median (middle quartile), the inter-quartile range (which represents the middle 50% of scores for the group), the upper quartile (75% of scores fall below the upper quartile), the lower quartile (25% of scores fall below the lower quartile). The whiskers show the 5th percentile and the 95th percentile respectively.

### Example 2: Diagnostic Performance of Biomarker S100A12 and Biomarker Combinations in Women with Endometriosis and Controls in Samples from a Multi-Center Study

The case group is comprised of patients diagnosed with peritoneal endometriosis, deep infiltrating endometriosis and endometriomas. Endometriosis (rASRM stages I-IV) was diagnosed by laparoscopic visualization with subsequent histological confirmation and the control group including healthy women without endometriosis. Inclusion criteria for the case group were the presence of pelvic pain/infertility and age between 18-45 years. Exclusion criteria for the case group were pregnant/breastfeeding, malignancy, recurrent endometriosis and laparoscopy/laparotomy ≤6 months.

S100A12 was measured with a pre-commercial ECLIA assay for S100A12, a sandwich-immunoassay which was developed for the cobas Elecsys® ECLIA platform (ECLIA Assay from Roche Diagnostics, Germany). The assay comprises a biotinylated and a ruthenylated monoclonal antibody that specifically binds S100A12. 6 μL were used from each serum sample and measured undiluted on a cobas e 801 analyzer (Roche Diagnostics, Germany). The Elecsys® Electro-ChemiLuminescence (ECL) technology and assay method is briefly described below for the determination of CA 125 II.

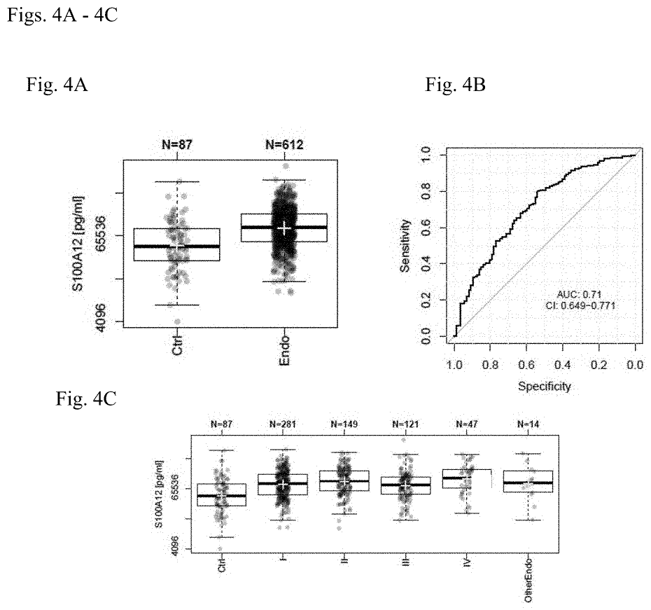

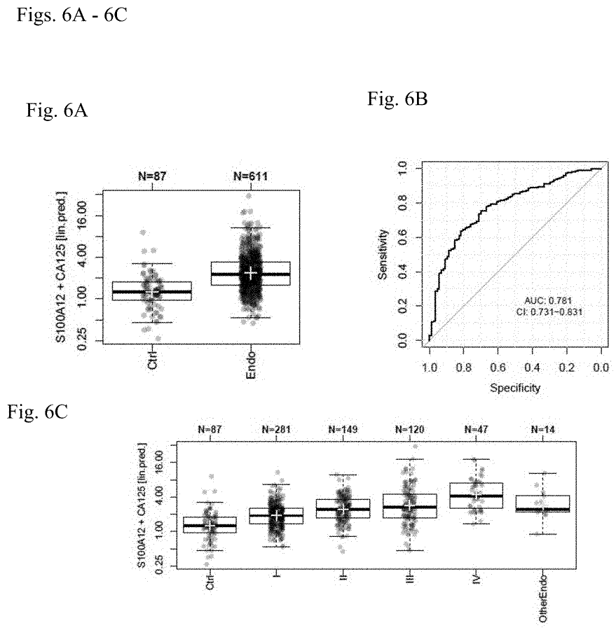

The concentration of CA-125 was determined by a cobas e 601 analyzer. Detection of CA 125 II with a cobas e 601 analyzer is based on the Elecsys® Electro-ChemiLuminescence (ECL) technology. In brief, biotin-labelled and ruthenium-labelled antibodies are combined with the respective amount of undiluted sample and incubated on the analyzer. Subsequently, streptavidin-coated magnetic microparticles are added and incubated on the instrument in order to facilitate binding of the biotin-labelled immunological complexes. After this incubation step the reaction mixture is transferred into the measuring cell where the beads are magnetically captured on the surface of an electrode. ProCell M Buffer containing tripropylamine (TPA) for the subsequent ECL reaction is then introduced into the measuring cell in order to separate bound immunoassay complexes from the free remaining particles. Induction of voltage between the working and the counter electrode then initiates the reaction leading to emission of photons by the ruthenium complexes as well as TPA. The resulting electrochemiluminescent signal is recorded by a photomultiplier and converted into numeric values indicating concentration level of the respective analyte. Receiver Operating Characteristic (ROC) curves were generated (see FIGS. 4A-C, 5A-5C, 7A-7C and 8A-8C for single biomarkers and FIGS. 6A-6C for biomarker combinations). The model performance is determined by looking at the area under the curve (AUC).

TABLE 3
Diagnostic performance of single biomarkers S100A12
and CA-125 and biomarker combinations in endometriosis
cases (rASRM stages I-IV) and controls.

| | | p-value (Mann- | N | N |
|---|---|---|---|---|
| Biomarker | AUC | Whitney test) | (cases) | (controls) |
| S100A12 | 0.71 | 0 | 612 | 87 |
| | (0.65-0.77) | | | |
| CA-125 | 0.72 | 0 | 616 | 88 |
| | (0.67-0.77) | | | |
| S100A12 + | 0.78 | 0 | 611 | 87 |
| CA-125 | (0.73-0.83) | | | |

For the multivariate analysis, a cut-off for the combination of the different biomarkers was applied to predict endometriosis based on multivariable logistic regression analysis.

Multivariable Logistic Regression Model with S100A12 and CA-125:

−0.223748351663371+1.26632635956222e-05*S100A12+0.0688766536162976*CA125

As shown in the table above, the AUC for S100A12 in Example 2 is different from that reported in Example 1. In Example 1 the respective reported AUC is 0.9167 (0.7825-1.0000) whereas in Example 2, the reported AUC for S100A12 is 0.71 (0.65-0.77). This difference can be attributed to the following reasons: the sample size of the cohort used in Example 2 is higher compared to that analysed in Example 1. Additionally, the cohort of Example 2 consists of samples collected in multiple study centers, as opposed to the cohort of Example 1 which contains samples collected from one single study site. Therefore, the cohort of Example 2 is more heterogeneous. Another reason that could explain the difference in the estimated AUC between the two cohorts is the use of different assays to detect S100A12 which were used to measure the cohorts. The measurements of the cohort in Example 2 were done using a pre-commercial automated ECLIA assay, run on an automated cobas Elecsys® ECLIA platform whereas the measurements of the cohort shown in Example 1 were performed using a commercial ELISA assay.

FIGS. 4A-4C. Box plots and ROC curves of serum levels of S100A12 in endometriosis cases (rASRM stages I-IV) and controls. (FIG. 4A) The box plot of serum S100A12 levels of controls versus endometriosis cases (rASRM stages I-IV). The lower and upper edges of each box correspond to the first and third quartile, respectively. The middle thick line represents the median value and the upper and lower whiskers represent values that are 1.5 times the interquartile range. (FIG. 4B) The ROC curve for serum S100A12 is shown. (FIG. 4C) Box plots for serum S100A12 levels of controls versus endometriosis rASRM stage I, II, III and IV respectively.

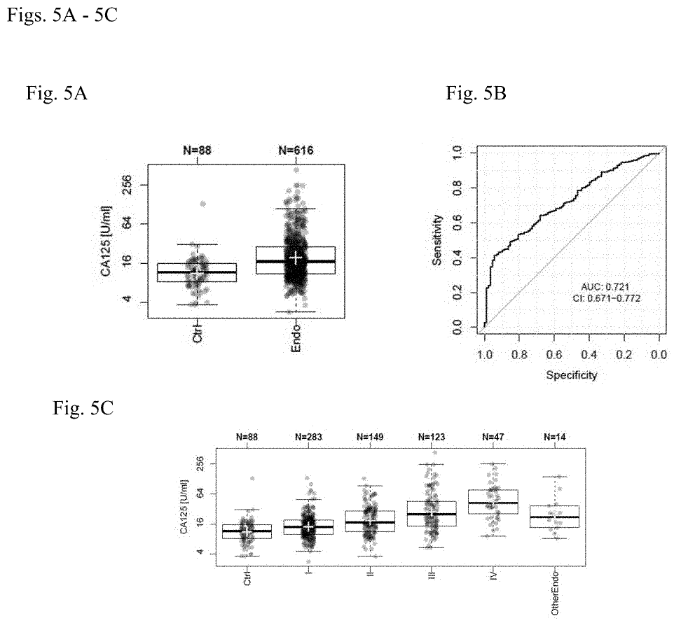

FIGS. 5A-5C. Box plots and ROC curves of serum levels of CA-125 in endometriosis cases (rASRM stages I-IV) and controls. (FIG. 5A) The box plot shows serum CA-125 levels of controls versus endometriosis cases (rASRM stages I-IV). The lower and upper edges of each box correspond to the first and third quartile, respectively. The middle thick line represents the median value and the upper and lower whiskers represent values that are 1.5 times the interquartile range. (FIG. 5B) The ROC curve for serum CA-125 is shown. (FIG. 5C) Box plots for serum CA-125 levels of controls versus endometriosis rASRM stage I, II, III and IV respectively.

FIGS. 6A-6C. Box plots and ROC curves of combination of serum levels of S100A12 and CA-125 in endometriosis cases (rASRM stages I-IV) and controls. (FIG. 6A) Box plot analysis for biomarker combination, serum S100A12 and CA-125, in controls and endometriosis cases (rASRM stages I-IV). The lower and upper edges of each box correspond to the first and third quartile, respectively. The middle thick line represents the median value and the upper and lower whiskers represent values that are 1.5 times the interquartile range. (FIG. 6B) The ROC curve for combination of serum S100A12 and CA-125 is shown. (FIG. 6C) Box plots for combination of serum S100A12 and serum CA-125 levels of controls versus endometriosis rASRM stage I, II, III and IV respectively.

TABLE 4
Diagnostic performance of single biomarkers S100A12 and CA-
125 in endometriosis cases (rASRM stages I-II) and controls.

| | | p-value (Mann- | N | N |
|---|---|---|---|---|
| Biomarker | AUC | Whitney test) | (cases) | (controls) |
| S100A12 | 0.71 | 0 | 430 | 87 |
| | (0.65-0.77) | | | |
| CA-125 | 0.67 | 0 | 432 | 88 |
| | (0.61-0.72) | | | |

The diagnostic performance of S100A12 to distinguish endometriosis cases (rASRM stages I-II) versus controls is higher compared to the diagnostic performance of the biomarker CA-125.

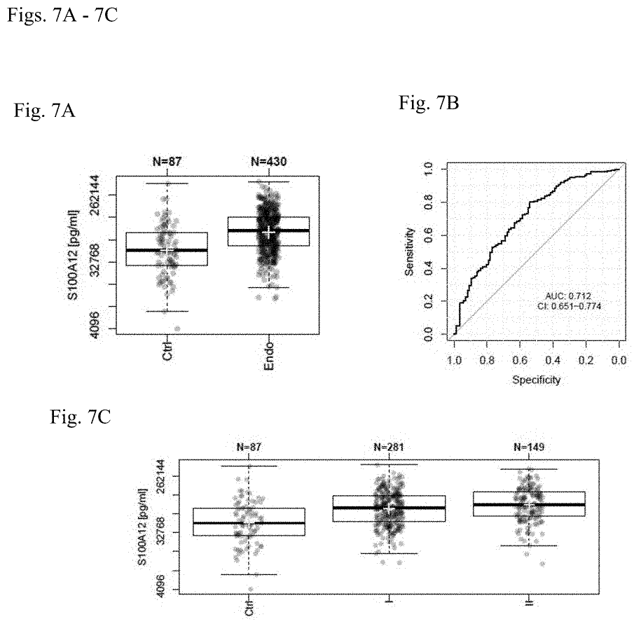

FIGS. 7A-7C. Box plots and ROC curves of serum levels of S100A12 in endometriosis cases (rASRM stages I-II) and controls. (FIG. 7A) The box plot shows serum S100A12 levels of controls versus early endometriosis cases (rASRM stages I-II). The lower and upper edges of each box correspond to the first and third quartile, respectively. The middle thick line represents the median value and the upper and lower whiskers represent values that are 1.5 times the interquartile range. (FIG. 7B) The ROC curve for serum S100A12 is shown for controls and cases (rASRM stages I-II). (FIG. 7C) Box plots for serum S100A12 levels of controls versus endometriosis rASRM stages I and II respectively.

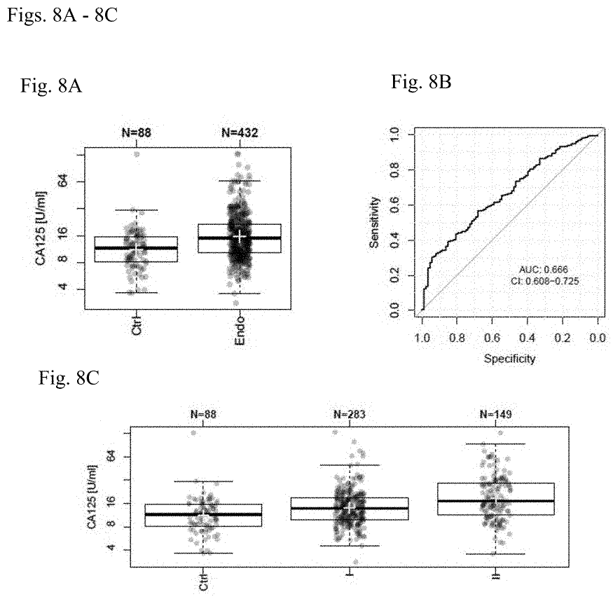

FIGS. 8A-8C. Box plots and ROC curves of serum levels of CA-125 in endometriosis cases (rASRM stages I-II) and controls. (FIG. 8A) The box plot shows serum CA-125 levels of controls versus early endometriosis cases (rASRM stages I-II). The lower and upper edges of each box correspond to the first and third quartile, respectively. The middle thick line represents the median value and the upper and lower whiskers represent values that are 1.5 times the interquartile range. (FIG. 8B) The ROC curve for serum CA-125 is shown for controls and cases (rASRM stages I-II). (FIG. 8C) Box plots for serum CA-125 levels of controls versus endometriosis rASRM stages I and II respectively.

### Example 3: Diagnostic Performance of Biomarker S100A12 and Biomarker Combinations in Women with Adenomyosis and Controls

The case group is comprised of patients diagnosed with adenomyosis by laparoscopic visualization with subsequent histological confirmation and the control group includes healthy women without adenomyosis. Inclusion criteria for the case group were the presence of pelvic pain/infertility and age between 18-45 years. Exclusion criteria of the case group were pregnant/breastfeeding, malignancy, recurrent adenomyosis and laparoscopy/laparotomy ≤6 months.

S100A12 was measured with a pre-commercial ECLIA assay for S100A12, a sandwich-immunoassay which was developed for the cobas Elecsys® ECLIA platform (ECLIA Assay from Roche Diagnostics, Germany). The assay comprises a biotinylated and a ruthenylated monoclonal antibody that specifically binds S100A12. 6 μL were used from each serum sample and measured undiluted on a cobas e 801 analyzer (Roche Diagnostics, Germany).

The concentration of CA-125 was determined by a cobas e 601 analyzer using the Elecsys® CA 125 II as described earlier in example 2.

Receiver Operating Characteristic (ROC) curves were generated by univariate models for the single biomarkers. The model performance is determined by looking at the area under the curve (AUC).

TABLE 5
Diagnostic performance of single biomarkers S100A12
and CA-125 in adenomyosis cases and controls.

| | | p-value (Mann- | N | N |
|---|---|---|---|---|
| Biomarker | AUC | Whitney test) | (cases) | (controls) |
| S100A12 | 0.71 | 2.33e−07 | 122 | 87 |
| | (0.64-0.78) | | | |
| CA-125 | 0.57 | 0.07429 | 124 | 88 |
| | (0.49-0.65) | | | |

The diagnostic performance of S100A12 to distinguish adenomyosis cases versus controls is higher compared to the diagnostic performance of the biomarker CA-125.

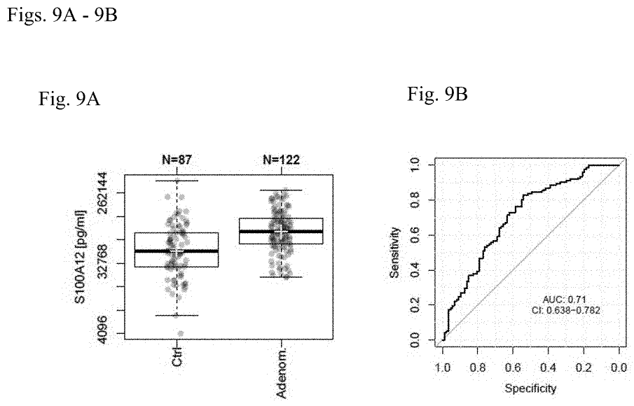

FIGS. 9A & 9B. Box plots and ROC curves of serum levels of S100A12 in adenomyosis cases and controls. (FIG. 9A) The box plot shows serum S100A12 levels of controls versus adenomyosis cases. The lower and upper edges of each box correspond to the first and third quartile, respectively. The middle thick line represents the median value and the upper and lower whiskers represent values that are 1.5 times the interquartile range. (FIG. 9B) The ROC curve for serum S100A12 is shown for controls and adenomyosis cases.

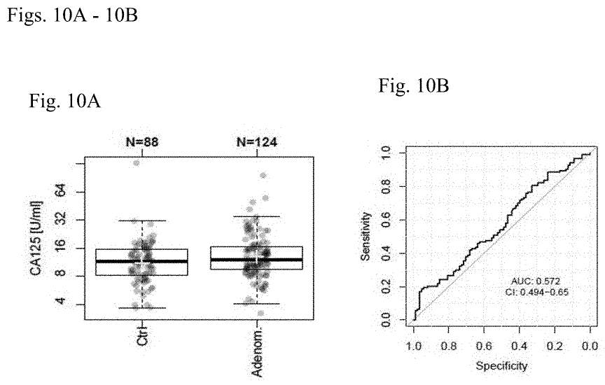

FIGS. 10A & 10B. Box plots and ROC curves of serum levels of CA-125 in adenomyosis cases and controls. (FIG. 10A) The box plot shows serum CA-125 levels of controls versus adenomyosis cases. The lower and upper edges of each box correspond to the first and third quartile, respectively. The middle thick line represents the median value and the upper and lower whiskers represent values that are 1.5 times the interquartile range. (FIG. 10B) The ROC curve for serum CA-125 is shown for controls and adenomyosis cases.

1. A method of assessing whether a patient has endometriosis or is at risk of developing endometriosis, comprising determining the amount or concentration of S100A12 in a sample of the patient, and comparing the determined amount or concentration to a reference.

2. A method of selecting a patient for drug-based therapy or surgical therapy (laparoscopy) of endometriosis, comprising determining the amount or concentration of S100A12 in a sample of the patient, and comparing the determined amount or concentration to a reference.

3. A method of monitoring a patient suffering from endometriosis or being treated for endometriosis, comprising determining the amount or concentration of S100A12 in a sample of the patient, and comparing the determined amount or concentration to a reference.

4. The method of claim 1, wherein an elevated amount or concentration of S100A12 in the sample of the patient is indicative of the presence of endometriosis in the patient.

5. The method of claim 1, wherein the sample is body fluid selected from the group consisting of blood, serum and plasma.

6. (canceled)

7. The method of claim 1, wherein the subject is a female patient, in particular a human female patient.

8. The method of claim 1, wherein the assessment is performed independent of the rASRM staging.

9. The method of claim 1, wherein endometriosis is selected from the group consisting of stage I endometriosis according to rASRM staging, stage II endometriosis according to rASRM staging, stage III endometriosis according to rASRM staging, and stage IV endometriosis according to rASRM staging.

10. The method of claim 1, wherein endometriosis is stage I endometriosis according to rASRM staging or stage II endometriosis according to rASRM staging.

11. The method of claim 1, wherein endometriosis is selected from the group consisting of peritoneal endometriosis, endometrioma, deep infiltrating endometriosis, and adenomyosis.

12. The method of claim 1, wherein preferable adenomyosis is assessed.

13. The method of claim 1, further comprising the assessment of dysmenorrhea according to the VAS scale and/or lower abdominal pain according to the VAS scale.

14. The method of claim 1, further comprising determining the amount or concentration of CA-125.

15. The method of claim 13, comprising calculating a ratio of the amount or concentration of S100A12 and dysmenorrhea and/or of the amount or concentration of S100A12 and lower abdominal pain according to the VAS scale.

16. The method of claim 14, comprising calculating a ratio of the amount or concentration of S100A6 and the amount or concentration of CA-125.
